(19) United States
(12) Patent Application Publication
(10) Pub. No.: US 2012/0059174 A1
(43) Pub. Date: Mar. 8, 2012

(54) PROCESS FOR PREPARING EPSILON-CAPROLACTONE AND 1,6-HEXANEDIOL

(75) Inventors: Olivier ABILLARD, Mannheim (DE); Daniel BREUNINGER, Bobenheim-Roxheim (DE); Eva KRETZSCHMAR, Mannheim (DE); Rolf PINKOS, Bad Dürkheim (DE)

(73) Assignee: BASF SE, Ludwigshafen (DE)

(21) Appl. No.: 13/226,049

(22) Filed: Sep. 6, 2011

Related U.S. Application Data
(60) Provisional application No. 61/380,731, filed on Sep. 8, 2010.

(51) Int. Cl. C07D 313/04

(52) U.S. Cl. 549/266

(57) ABSTRACT

The invention relates to processes for preparing 1,6-hexanediol and very pure ε-caprolactone from a dicarboxylic acid solution (DCS), comprising the steps of (a) esterification of the DCS with alcohols, (b) partial catalytic hydrogenation of the esters, (c) distillative removal of 1,6-hexanediol and low boilers as the top product, and (d) cyclization of the 6-hydroxycaproic ester present in the bottoms fraction in the presence of a higher-boiling alcohol than caprolactone.

This patent application claims the benefit of pending U.S. provisional patent application Ser. No. 61/380,731 filed Sep. 8, 2010 incorporated in its entirety herein by reference.

The present invention relates to a process for preparing ε-caprolactone having at least 99% purity, and for preparing 1,6-hexanediol, from a dicarboxylic acid solution (DCS), comprising the steps of (a) esterification of the DCS with alcohol, (b) partial catalytic hydrogenation of the esters, (c) distillative removal of the 1,6-hexanediol and (d) cyclization of the 6-hydroxycaproic ester present in the bottoms fraction, said process being performed in such a way that the cyclization takes place in the presence of a higher-boiling alcohol relative to ε-caprolactone. More particularly, the invention relates to a process in which the higher-boiling alcohol relative to ε-caprolactone is used at the early stage of esterification of the DCS (step (a)), and to a process in which the conditions of the hydrogenation (step (b)) and/or of the subsequent distillation (step (c)) are selected such that 1,6-hexanediol formed in the process displaces lower-boiling alcohols originally used for esterification by transesterification. The invention further relates to embodiments of this process in which the 1,6-hexanediol from step (c) and/or the ε-caprolactone from step (d) are purified further by distillation.

ε-Caprolactone and the polycaprolactones prepared therefrom by polyaddition serve for preparation of polyurethanes. 1,6-Hexanediol is a monomer unit which is used predominantly in the polyester and polyurethane sector.

Integration with production plants for preparation of cyclohexanol and cyclohexanone and the utilization of the dicarboxylic acid solution (DCS) waste product as a starting material for the preparation of ε-caprolactone and 1,6-hexanediol leads to favorable feedstock costs compared to pure 6-hydroxycaproic acid and pure adipic acid. It also constitutes an environmentally friendly utilization of a waste product.

The aqueous dicarboxylic acid solution (DCS) which forms as a by-product in the oxidation of cyclohexane to cyclohexanol and cyclohexanone (cf. Ullmann's Encyclopedia of Industrial Chemistry, 5th ed., 1987, vol. A8, p. 219) comprises (calculated without water in % by weight) generally between 10 and 40% adipic acid, between 10 and 60% 6-hydroxycaproic acid, between 1 and 10% glutaric acid, between 1 and 10% 5-hydroxyvaleric acid, between 0.5 and 5% 5-formylvaleric acid, between 1 and 5% 1,2-cyclohexanediols, between 1 and 5% 1,4-cyclohexanediols, between 2 and 10% formic acid, and a multitude of further mono- and dicarboxylic acids, esters, oxo and oxa compounds, the individual contents of which generally do not exceed 5%. Examples of mono- and dicarboxylic acids, esters, oxo and oxa compounds include acetic acid, propionic acid, butyric acid, valeric acid, caproic acid, oxalic acid, malonic acid, dihydromuconic acid, succinic acid, 4-hydroxybutyric acid, γ-butyrolactone and ε-caprolactone. It is desirable to use DCS for the production of 1,6-hexanediol and pure ε-caprolactone proceeding from the 6-hydroxycaproic acid and adipic acid present in the DCS.

The existing prior art shows that adipic diesters prepared from DCS and monoalcohols can be hydrogenated to 1,6-hexanediol with high yields of about 96 to 99%. The catalysts used for the hydrogenation were based on copper and zinc oxide, copper and aluminum oxide, or copper, aluminum oxide and zinc oxide.

The preparation of caprolactone from DCS has already been described, for example, in DE 1 618 143. In this case, dewatered DCS is reacted thermally with phosphoric acid, and a mixture of dicarboxylic acids, caprolactone and a multitude of other components is fractionated. The bottoms are in some cases obtained in solid and sparingly soluble form. However, the caprolactone even after further distillative workup has only 98% purity.

There have also been many descriptions of conversion of 6-hydroxycaproic acid or esters thereof to caprolactone (e.g. DE 2 013 525, EP-A 349 861 and literature cited therein).

The working example of DE 196 07 954 describes esterifying dewatered DCS with methanol and separating the ester mixture, after removal of low boilers, into a dimethyl adipate fraction and a methyl hydroxycaproate fraction. The dimethyl adipate fraction was accordingly hydrogenated in the presence of catalysts comprising copper, zinc oxide and aluminum oxide to give 1,6-hexanediol and methanol, and the ester conversion at 220° C. and 220 bar was 99.5%, the 1,6-hexanediol selectivity >99%. The methyl hydroxycaproate fraction was cyclized catalytically to caprolactone, and the purity after subsequent distillation was 99.9%. In order that, when methanol or other low-boiling alcohols are selected for the esterification of the DCS, the 6-hydroxycaproic ester can be cyclized to the caprolactone in a satisfactory yield, the adipic ester has to be removed completely from the 6-hydroxycaproic ester fraction before the cyclization. In order to ensure this, a high level of separation complexity is firstly required, and it is secondly necessary to employ a method in which a portion of the 6-hydroxycaproic ester is removed together with the adipic ester and is thus lost to the cyclization. The use of higher-boiling alcohols for the esterification of the DCS in this process would complicate the separation of the adipic ester fraction and the 6-hydroxycaproic ester fraction. Another disadvantage of this process involving the use of methanol or other low-boiling alcohols for the esterification of the DCS is that the esterification has to be performed at comparatively low temperatures (<200° C.) to avoid very high pressures, which is why it is usually necessary to use a catalyst, generally sulfuric acid, in order to ensure effective esterification. In these cases, however, the sulfuric acid has to be removed again in a complex manner after the esterification. In addition, the removal of water of reaction in the case of use of low-boiling alcohols is possible only with a very high level of complexity, and so it is not done in practice. The presence of water in the esterification causes relatively poor conversions, such that relatively small amounts of acid are still present in the esterification output. These lead, in later process stages (particularly in distillations), to the formation of particularly polyhydroxycaproic esters in the bottoms fractions. These either have to be worked up and recycled in a complex manner, or are lost to the further processing to give caprolactone or hexanediol.

In addition, DE 196 07 954 describes a process variant (“variant E”) especially for the preparation of small amounts of caprolactone with minimal technical complexity. Accordingly, an alcohol having a higher boiling point than caprolactone should be used for the esterification of the DCS, and the reaction is performed, without isolation of the adipic ester fraction, batchwise in a one-pot reaction in the presence of an esterification catalyst. The caprolactone can then be obtained as the distillate, while the high-boiling adipic ester remains in the bottoms. Disadvantages here are the batchwise process regime and only a moderate purity of the caprolactone thus obtainable (approx. 98%).

The problem underlying the invention can therefore be considered that of preparing 1,6-hexanediol and very pure ε-caprolactone proceeding from DCS, while avoiding the disadvantages of the prior art, i.e. either high preparation costs or insufficient purity and yield of the products. This object is achieved by the embodiments described hereinafter and claimed.

Accordingly, the present invention relates to a process for preparing 1,6-hexanediol and very pure ε-caprolactone from a dicarboxylic acid solution (DCS), comprising the steps of
(a) esterification of the DCS with alcohols,
(b) partial catalytic hydrogenation of the esters,
(c) distillative removal of the 1,6-hexanediol as the top product, and
(d) cyclization of the 6-hydroxycaproic ester present in the bottoms fraction to ε-caprolactone in the presence of an alcohol having a boiling point greater than that of ε-caprolactone.

More particularly, the present invention relates to a process for preparing 1,6-hexanediol and very pure ε-caprolactone from a dicarboxylic acid solution (DCS), comprising the steps of
(a) esterification of the DCS with alcohols,
(b) partial catalytic hydrogenation of the esters,
(c) distillative removal of the 1,6-hexanediol as the top product, and
(d) cyclization of the 6-hydroxycaproic ester present in the bottoms fraction to ε-caprolactone in the presence of an alcohol having a boiling point greater than that of ε-caprolactone, wherein alcohols having a boiling point greater than that of ε-caprolactone are used for the early stage of esterification in step (a).

In addition, the present invention relates to a process for preparing 1,6-hexanediol and very pure ε-caprolactone from a dicarboxylic acid solution (DCS), comprising the steps of
(a) esterification of the DCS with alcohols,
(b) partial catalytic hydrogenation of the esters,
(c) distillative removal of the 1,6-hexanediol as the top product, and
(d) cyclization of the 6-hydroxycaproic ester present in the bottoms fraction to ε-caprolactone in the presence of an alcohol having a boiling point greater than that of ε-caprolactone; wherein the hydrogenation in step (b) and/or the distillation in step (c) are performed under conditions which enable displacement of any relatively low-boiling esterification alcohols from step (a) by the 1,6-hexanediol formed in the hydrogenation in the manner of a trans esterification. Such conditions are, for example, the use of hydrogenation catalyst having acidic or basic sites for the hydrogenation in step (b), the presence of small amounts of acids or bases (acid or base number in the feed to the distillation stage (c) of at least 0.01) in the distillation of step (c), and/or the presence of transesterification catalysts—e.g. sodium methoxide—(in amounts of at least 1 ppm) in the hydrogenation of step (b) and/or the distillation of step (c).

The process according to the invention for preparation of 1,6-hexanediol and very pure ε-caprolactone uses dicarboxylic acid solutions (DCS) as starting material. Such DCS are obtained in the form of aqueous solutions as by-products of the oxidation of cyclohexane to cyclohexanol and cyclohexanone (cf. Ullmann's Encyclopedia of Industrial Chemistry, 5th ed., 1987, vol. A8, p. 219). They comprise (calculated without water in % by weight) generally between 10 and 40% adipic acid, between 10 and 60% 6-hydroxycaproic acid, between 1 and 10% glutaric acid, between 1 and 10% 5-hydroxyvaleric acid, between 0.5 and 5% 5-formylvaleric acid, between 1 and 5% 1,2-cyclohexanediols, between 1 and 5% 1,4-cyclohexanediols, between 2 and 10% formic acid, and a multitude of further mono- and dicarboxylic acids, esters, oxo and oxa compounds, the individual contents of which generally do not exceed 5%. Examples of mono- and dicarboxylic acids, esters, oxo and oxa compounds include acetic acid, propionic acid, butyric acid, valeric acid, caproic acid, oxalic acid, malonic acid, dihydromuconic acid, succinic acid, 4-hydroxybutyric acid, γ-butyrolactone and caprolactone. The DCS is generally an aqueous solution having a water content of 20 to 80% by weight.

Aldehydes such as 5-formylvaleric acid and ketones such as 1,4-cyclohexanedione and 4-hydroxycyclohexanone can form acetals and ketals in the esterification with diols (step (a)). As a result, 5-formylvaleric acid can be lost to the preparation of ε-caprolactone and 1,6-hexanediol as a result of further reactions of the acetals. As a result of the ketal or acetal formation, some or all of the alcohol bound in each may be lost.

According to the composition of the DCS, it may therefore be advantageous to hydrogenate the aldehydes and ketones present catalytically to alcohols before the esterification step (a). If the cyclohexane oxidation has been performed in the absence of a deperoxidation catalyst, for example cobalt naphthenate, the DCS comprises 6-hydroperoxycaproic acid as described in DE-A 1 951 250 and EP-A 847 979. If oxidation has been effected in the presence of a deperoxidation catalyst, 6-hydroperoxycaproic acid is present only in small amounts. If the cyclohexane oxidation has been performed without catalyst, the 6-hydroperoxycaproic acid formed, just like 5-formylvaleric acid, can be hydrogenated to 6-hydroxycaproic acid. This optional hydrogenation then takes place before step (a) of the process according to the invention.

Since a hydroperoxy group in one case and an aldehyde group in another case has to be hydrogenated in the hydrogenation which optionally takes place before step (a) of the process according to the invention, the optimal hydrogenation conditions of the two compounds differ. Since the hydroperoxycaproic acid can also be converted purely thermally, but less selectively than in a hydrogenation, to 6-hydroxycaproic acid, it is hydrogenated according to DE-A 1 951 250 in the presence of palladium, rhodium or platinum catalysts at 15 to 130° C., preferably 50 to 100° C., i.e. at moderate temperatures. Keto and aldehyde groups are not hydrogenated under the conditions of the 6-hydroperoxycaproic acid hydrogenation in DE-A 1 951 250. Higher temperatures and pressures are needed for that purpose.

The hydrogenation which is optionally performed before step (a) of the process according to the invention is effected at 10 to 200° C., preferably 30 to 180° C., more preferably 50 to 170° C. The partial hydrogen pressure is 1 to 100 bar, preferably 10 to 80 bar, more preferably 30 to 60 bar.

For the catalytic hydrogenation which is optionally performed before step (a) of the process according to the invention, the catalysts used comprise at least one metal of groups 7 to 12 of the Periodic Table, for example ruthenium, palladium, rhodium, nickel, cobalt, iron, rhenium, platinum, iridium, copper, osmium and zinc. Also very suitable are unsupported catalysts which do not comprise any support and consist of metals, metal oxides or mixtures thereof. Preference is given to unsupported iron and especially cobalt catalysts.

For the esterification in step (a) of the process according to the invention to esterify the carboxylic acids present in the DCS, it is possible to use alcohols having 1 to 30 carbon atoms, preference being given to those with a higher boiling point than ε-caprolactone within the pressure range from 10 to 1500 mbar (for example 96-97° C. at 20 mbar or 235° C. at 1013 mbar). It is possible to use monoalcohols, and also diols and other polyhydric alcohols. It is equally possible to use mixtures of such alcohols or compositions which comprise such alcohols (preferably in a proportion of at least 30% by weight, more preferably in a proportion of at least 50% by weight). Examples of such alcohols are glycerol, trimethylolpropane, 1,6-hexanediol, 1,8-octanediol, 1,10-decanediol, 1,12-dodecanediol, 1-tridecanol, 1-tetradecanol, 1-pentadecanol, 1-hexadecanol, 1-octadecanol, 1-eicosanol, butylethylpropanediol, neopentyl glycol, pentaerythritol, triethylene glycol, tetraethylene glycol, bis(6-hydroxyhexyl)ether. Particularly suitable are polyhydric alcohols, especially diols, especially α,ω-diols having six to twelve carbon atoms. Particular preference is given to 1,6-hexanediol, since this esterification alcohol corresponds to a target product of the process according to the invention.

The use of alcohols with higher boiling points than ε-caprolactone for the inventive esterification of the DCS is advantageous in order to achieve a satisfactory conversion of the 6-hydroxycaproic ester in the cyclization step (d) of the process according to the invention in the presence of adipic acid or adipic ester. When the esterification alcohol used has been a low-boiling alcohol, for example methanol, the process according to the invention is likewise employable, provided that hydrogenation step (b) and/or distillation step (c) are performed in such a way that 1,6-hexanediol formed in the hydrogenation, as a higher-boiling alcohol than caprolactone, displaces the original esterification alcohol as low boilers from esters thereof and hence is present in the cyclization step (d), such that the cyclization can proceed effectively in the presence of adipic acid equivalents.

In order that the hexanediol formed can displace the low-boiling alcohol from esters thereof during the hydrogenation and subsequent distillation, certain prerequisites have to be met. The hydrogenation catalyst should possess acidic or basic sites, either Lewis and/or Brønsted sites. Should the hydrogenation catalyst possess only an insufficient number of transesterifying catalytic sites, if any, a transesterification catalyst can be added to the hydrogenation feed, for example sodium methoxide. This additive is added in amounts of 1 to 1000 ppm based on the feed, preferably 5 to 500 ppm, more preferably 10 to 300 ppm. In order that hexanediol can release low-boiling alcohols from esters thereof in the course of distillation, acids or bases should be present in small amounts. This acid or base may, for example, already be a carboxylic acid such as adipic acid or adipic monoester or hydroxycaproic acid, the content of which causes an acid number of 0.01 to 5 in the feed to stage (c). In addition, additives such as sodium methoxide or titanates such as tetra-n-butyl titanate are of course also possible in amounts of 1 to 5000 ppm, preferably 5 to 3000 ppm, more preferably 10 to 2000 ppm.

In one embodiment of the process according to the invention, instead of pure 1,6-hexanediol, a portion of the hydrogenation output from step (b) is used for the esterification of the DCS in step (a). The hydrogenation output is an alcoholic composition which comprises generally 30 to 90% by weight of 1,6-hexanediol, 1 to 10% by weight of 1,5-pentanediol, 1,4-butanediol, 1,2-cyclohexanediol and 1,4-cyclohexanediol (in each case less than 5% by weight), and also up to 5% by weight of monoalcohols, for example n-butanol, n-pentanol and n-hexanol, and 1 to 50% by weight of oligomeric or polymeric high boilers compared to 1,6-hexanediol.

In a further embodiment of the process according to the invention, a portion of the top product from the distillation step (c) is used as the alcoholic composition for the esterification of the DCS in step (a).

The use of diols, especially of α,ω-diols, as esterification alcohols constitutes an advantage over monoalcohols. The losses of esterification alcohol are reduced and the workup of the hydrogenation output is simplified.

For the esterification in step (a) of the process according to the invention, preference is given to adding an alcohol with a higher boiling point than ε-caprolactone to the DCS, especially a corresponding α,ω-diol such as 1,6-hexanediol or a mixture of such alcohols or a composition comprising such alcohols (preferably having a proportion of such alcohols of at least 30% by weight, more preferably having a proportion of at least 50% by weight). Such a composition may be a portion of the hydrogenation output or a portion of the top product of the distillative removal (step (c)). The mass ratio of DCS to alcohol in the case of use of diols is preferably 1:0.2 to 1:0.8, especially 1:0.3 to 1:0.7 and more preferably 1:0.4 to 1:0.6. In the case of use of monoalcohols, the amount of alcohol should be increased by a factor of two; in the case of polyols, the amount of alcohol should be reduced correspondingly, for example by a factor of 1.5 for triols, by a factor of 2 for tetraols, a factor of 2.5 for pentaols, a factor of 3 for hexaols, etc. In the case of use of alcohol-comprising compositions for the esterification (for example hydrogenation output from step (b)), a corresponding increased amount—based on the alcohol content thereof—of the composition should be selected.

The removal of the water from the aqueous DCS and esterification in step (a) of the process according to the invention are preferably performed in one process step. For this purpose, it is possible to use stirred reactors, flow tubes and/or columns. Removal of water and esterification are preferably effected in at least one reactor with an attached distillation column. In order to achieve full conversion in the esterification of the carboxylic acids and full removal of water, 2 to 6, preferably 3 to 5, series-connected reactors with attached columns are employed.

The esterification reaction of the DCS in step (a) of the process according to the invention can proceed without addition of a catalyst. However, it is also possible to add a catalyst for the esterification to increase the reaction rate. This may be a homogeneous, dissolved or heterogeneous catalyst.

Examples of homogeneous catalysts for the esterification include sulfuric acid, phosphoric acid, hydrochloric acid, sulfonic acids such as p-toluenesulfonic acid, heteropolyacids such as tungstophosphoric acid, or Lewis acids, for example compounds of aluminum, vanadium, titanium or boron. Preference is given to mineral acids, especially sulfuric acid. The weight ratio of homogeneous catalyst to carboxylic acid is generally 0.0001 to 0.5, preferably 0.001 to 0.3.

Suitable heterogeneous catalysts are acidic or superacidic materials, for example acidic and superacidic metal oxides such as SiO₂, Al₂O₃, SnO₂, ZrO₂, sheet silicates or zeolites, all of which may be doped with mineral acid residues such as sulfate or phosphate to increase the acid strength, or organic ion exchangers with sulfonic acid or carboxylic acid groups. The solid catalysts may be arranged as a fixed bed or used in the form of a suspension.

Preference is given to esterifying without addition of catalyst.

The bottom temperature in the reactors with attached column is 200 to 250° C. The esterification is effected with simultaneous removal of the water present in the DCS and of the water of reaction. The esterification and removal of water can be performed at pressures of 0.1 to 5 bar, preferably 0.5 to 3 bar, more preferably at 0.8 to 1.3 bar. The residence time, calculated over all to 10 hours. The esterification can be performed continuously or batchwise.

The top product obtained from the attached columns is the water present in the DCS and that formed in the esterification. The top product may further comprise organic by-products, for example lower monocarboxylic acids, e.g. formic acid.

The bottom product obtained in the last reactor is an ester mixture (in the case of use of diols, a mixture of oligo- and polyesters) which was formed from the carboxylic acids present in the DCS, the cyclohexanediols and the added alcohols. Also present in the bottom product are unconverted alcohols. This mixture is used for the subsequent catalytic hydrogenation in step (b) of the process according to the invention.

The completeness of the conversion of the free carboxyl groups present in the carboxylic acid mixture is determined by the acid number measured after the esterification (mg KOH/g). Minus any acid added as a catalyst it is 1 to 20, preferably 2 to 15 and more preferably 5 to 10 mg KOH/g.

If a dissolved acid has been used as a catalyst for the esterification, the ester mixture is appropriately neutralized with a base, in which case 1 to 1.5 base equivalents are added per acid equivalent of the catalyst. The bases used are generally alkali metal or alkaline earth metal oxides, carbonates, hydroxides or alkoxides, or amines in substance or dissolved in the esterification alcohol. The acid added can also be removed by passing the ester mixture through a weakly basic ion exchanger.

The hydrogenation of the ester mixture in step (b) of the process according to the invention is effected catalytically in the liquid phase in the presence of fixed bed or suspended catalysts, preferably fixed bed catalysts. The temperatures employed are between 100 and 350° C., preferably 120 and 300° C., more preferably 140 and 280° C., and pressures of 30 to 350 bar, preferably 40 to 320 bar, more preferably 50 to 300 bar. The catalyst hourly space velocity is 0.2 to 1.5 kg of ester per kg of catalyst and hour.

According to the invention, the hydrogenation in step (b) is effected partially, i.e. in at least a portion, but not completely.

The conversion in the hydrogenation in step (b) of the process according to the invention is deliberately restricted in order to ensure a higher proportion of 6-hydroxycaproic esters for the cyclization to ε-caprolactone (step (d)). Preference is given to establishing a partial hydrogenation with a hydrogenation conversion in the range from 40 to 97%, more preferably of 50 to 95%. This can be accomplished, for example, by altering the hydrogenation temperature, increasing the space velocity, lowering the pressure, or in principle also by bypassing the postreactor.

The hydrogenation conversion is defined as the ratio of the reduction in the ester number (EN) in the reaction mixture during the hydrogenation to the total ester number in the reaction mixture before the hydrogenation:

H%=100*(EN_{R,B}−EN_{R,A})/EN_{R,B}
EN_{R,B}: Ester number of the reaction mixture before the hydrogenation
EN_{R,A}: Ester number of the reaction mixture after the hydrogenation
H %: Hydrogenation conversion in percent

The ester number (EN) is determined from the difference between the hydrolysis number (HN) and the acid number (AN) of the mixture:

EN_{R}=HN_{R}−AN_{R}
EN_{R}: Ester number of the particular reaction mixture
HN_{R}: Hydrolysis number of the particular reaction mixture
AN_{R}: Acid number of the particular reaction mixture

The hydrolysis number (HN) is determined to DIN 53401 and the acid number (AN) to DIN 54302 (new version of DIN EN ISO 2114).

Alternatively, the hydrogenation conversion can also be determined by other methods, for instance from the consumption of hydrogen in the reaction mixture.

The hydrogenation conversion in step (b) of the process according to the invention can be adjusted to the desired value for the particular starting conditions (nature of the plant, of the catalyst and of the starting substances). If too high a hydrogenation conversion is found, it can be reduced by stepwise lowering of the hydrogenation temperature, of the reactor pressure or of the amount of hydrogen supplied per unit time, or by increasing the feed rate per unit time. Conversely, if too low a hydrogenation conversion is found, it can be increased by stepwise increase in the hydrogenation temperature, in the reactor pressure, or in the amount of hydrogen supplied per unit time, or by lowering the feed rate per unit time. It is possible to undertake a single alteration or else two or more thereof. After each alteration, the newly established hydrogenation conversion has to be determined after a sufficient time and, if appropriate, further alterations have to be made.

The hydrogenation in step (b) of the process according to the invention can in principle be performed in only one reactor. However, this method has disadvantages: ester hydrogenations are strongly exothermic and additionally have to be performed at high temperatures. For instance, the hydrogenation temperature according to U.S. Pat. No. 3,524,892, where oligoesters prepared from DCS were hydrogenated in the presence of barium oxide-doped copper chromite, is 260 to 270° C. Reliable removal of heat from the reactor requires a high level of complexity. The hydrogenation is therefore preferably performed in at least two reactors connected in series. When fixed bed catalysts are employed, the hydrogenation feed can be passed over the catalyst in liquid phase mode or trickle mode. When working in liquid phase mode, hydrogen gas is introduced into the reactor flooded with the liquid reaction mixture, and the hydrogen passes through the catalyst bed in ascending gas bubbles. When working in trickle mode, the liquid ester mixture is allowed to trickle over the catalyst bed arranged in the reactor which is under hydrogen pressure, forming a thin liquid film on the catalyst. In a preferred embodiment, two or more reactors are used, in which case the predominant portion of the esters is hydrogenated in the first reactor. The first reactor is preferably operated with liquid circulation for heat removal by means of heat exchangers, and the downstream reactor(s) preferably in straight pass, without circulation, to complete the conversion. This mode of operation is referred to as circulation mode.

The hydrogenation in step (b) of the process according to the invention can be effected batchwise or continuously.

The hydrogenation in step (b) of the process according to the invention is generally performed with the ester mixture which comprises excess alcohols and is obtained in the esterification without additional solvent. However, it may also be advantageous to work in the presence of a solvent which is inert under the reaction conditions. Useful solvents include, for example, all alcohols used for the esterification, and also tetrahydrofuran, dioxane and monoalcohols having 1 to 6 carbon atoms, for example methanol, ethanol, propanols, n-butanol, n-hexanol or mixtures of the compounds mentioned. The amount of solvent is 5 to 50% by weight, preferably 10 to 30% by weight, based on the ester mixture. Preference is given to performing the hydrogenation without solvent.

It may also be advantageous to meter a base into the ester mixture obtained in the esterification. The bases used are preferably lithium alkoxides, sodium alkoxides and potassium alkoxides, more preferably sodium methoxide. The amount of base is 20 to 180 ppm, preferably 30 to 90 ppm, based on the ester mixture. In an ester mixture with a residual acid number of >1 mg KOH/g, the residual acids are neutralized only in insignificant amounts. The base added serves to suppress the formation of by-products which might otherwise form in the hydrogenation, for example hexanol or ether compounds.

The hydrogenation in step (b) of the process according to the invention is effected in the presence of a catalyst. Useful catalysts in principle are all homogeneous and heterogeneous catalysts suitable for hydrogenation of carbonyl groups, such as metals, metal oxides, metal compounds or mixtures thereof. Examples of homogeneous catalysts are described in H. Kropf, Houben-Weyl, Methoden der Organischen Chemie [Methods of Organic Chemistry], volume IV/Ic, Georg Thieme Verlag Stuttgart, 1980, p. 45 to 67, and examples of heterogeneous catalysts are described in Houben-Weyl, Methoden der Organischen Chemie, volume IV/Ic, p. 16 to 26.

Preference is given to using catalysts which comprise one or more of the elements from transition groups I and VI to VIII of the Periodic Table of the Elements, preferably copper, chromium, molybdenum, manganese, rhenium, ruthenium, cobalt, nickel and palladium, more preferably copper, cobalt or rhenium.

The catalysts may consist solely of the active components, or the active components may be applied to supports. Suitable support materials are, for example, Cr₂O₃, Al₂O₃, SiO₂, ZrO₂, TiO₂, ZnO₂, BaO and MgO, or mixtures thereof.

Preference is given to catalysts as described in EP 0 552 463. These are catalysts which, in the oxidic form, have the composition CuₐAl_{b}Zr_{c}Mn_{d}Oₓ where a>0, b>0, c≧0, d>0, a>b/2, b>a/4, a>c, a>d and x is the number of oxygen ions required per formula unit to ensure electronic neutrality. These catalysts can be prepared, for example, according to EP 0 552 463 by precipitating sparingly soluble compounds from solutions which comprise the corresponding metal ions in the form of salts thereof. Suitable salts are, for example, halides, sulfates and nitrates. Suitable precipitants are all agents which lead to the formation of those insoluble intermediates which can be converted to the oxides by thermal treatment. Particularly suitable intermediates are the hydroxides and carbonates or hydrogencarbonates, and so alkali metal carbonates or ammonium carbonates are used as particularly preferred precipitants. What is important for the preparation of the catalysts is the thermal treatment of the intermediates at temperatures between 500° C. and 1000° C. The BET surface area of the catalysts is between 10 and 150 m²/g.

In one embodiment, the catalysts used for the hydrogenation in step (b) of the process according to the invention have shaped body precursors which comprise, in addition to copper oxide, aluminum oxide and at least one of the oxides of lanthanum, of tungsten, of molybdenum, of titanium, of zirconium or of iron, also metallic copper, copper flakes, pulverulent cement, graphite or a mixture as already described in the process for hydrogenation of the oligo- and polyesters of DCS with diols. The catalyst and preparation thereof are described in WO 2004/085356, WO 2006/005505 and WO 2007/006719.

In one embodiment, the hydrogenation in step (b) of the process according to the invention is performed by means of a shaped catalyst body, the precursor of which is producible by
(i) providing an oxidic material comprising copper oxide, aluminum oxide and at least one of the oxides of lanthanum, of tungsten, of molybdenum, of titanium, of zirconium or of iron,
   (ii) adding pulverulent metallic copper, copper flakes, pulverulent cement, graphite or a mixture thereof to the oxidic material and
   (iii) shaping the mixture resulting from step (ii) to a shaped body.

Especially the hydrogenation of oligomeric esters of different composition, as form in the inventive esterification of a DCS with diols or diol mixtures, places high demands on the particular catalyst, since it must not be poisoned and nevertheless has to exhibit a high activity and selectivity. The above-described catalysts which have shaped body precursors comprising, in addition to copper oxide, aluminum oxide and at least one of the oxides of lanthanum, of tungsten, of molybdenum, of titanium, of zirconium or of iron, also metallic copper, copper flakes, pulverulent cement, graphite or a mixture were found to be particularly suitable for this application.

Before use as a catalyst, the shaped bodies are activated in a manner known per se by treatment with reducing media, for example hydrogen. The activation is effected either beforehand in a reduction oven or after installation in the reactor. If the catalyst precursor has already been activated beforehand in a reduction oven, it is installed into the reactor and contacted directly with the hydrogenation solution under hydrogen pressure.

The hydrogenation output of step (b) generally has a composition of 30-90% by weight of 1,6-hexanediol, 1-10% by weight of 1,5-pentanediol, <5% 1,4-butanediol, <5% 1,2-cyclohexanediol, <5% 1,4-cyclohexanediol, <5% monoalcohols and 1-50% by weight of oligomeric or polymeric esters of adipic acid and 6-hydroxycarboxylic acid (high boilers compared to 1,6-hexanediol). The composition depends more particularly on the establishment of the hydrogenation conversion (should be established in accordance with the invention between 40 and 97%, preferably between 50 and 95%). A comparatively high hydrogenation conversion should be established if maximum amounts of 1,6-hexanediol are to be obtained in the process according to the invention. A comparatively low hydrogenation conversion should be established when maximum amounts of pure ε-caprolactone are to be prepared in the process according to the invention. In the case of a full hydrogenation, all 6-hydroxycaproic ester would be hydrogenated and therefore no longer be available as starting material for the cyclization to ε-caprolactone. On the other hand, the hydrogenation step and the subsequent distillative removal of the 1,6-hexanediol formed are necessary, since 1,2-cyclohexanediol is also removed efficiently in this way before the cyclization reaction, thus allowing very pure and virtually 1,2-cyclohexanediol-free ε-caprolactone to be obtained (less than 0.05% by weight of 1,2-cyclohexanediol in the remaining ester stream).

In one embodiment of the process according to the invention, a portion of the hydrogenation output, which comprises a high proportion of 1,6-hexanediol, is used for the esterification of the DCS directly or after distillative purification, instead of pure 1,6-hexanediol. The advantage of this process is that other diols such as 1,5-pentanediol, 1,4-butanediol, 1,4-cyclohexanediol, 1,2-cyclohexanediol, some of which constitute by-products, replace the 1,6-hexanediol. This reduces losses of 1,6-hexanediol, and the hydrogenation stream to be worked up is reduced in size.

In distillation step (c) of the process according to the invention, diols such as 1,6-hexanediol, 1,5-pentanediol, 1,4-butanediol, 1,4-cyclohexanediol, 1,2-cyclohexanediol and various low boilers are removed in a first column from the hydrogenation output from step (b). The column has 1 to 30 theoretical plates. The bottom temperatures employed are 120 to 250° C., and the pressures employed from 5 to 500 mbar.

The top product from this distillation step (c) comprises generally 75 to 95% by weight of 1,6-hexanediol, 3 to 10% by weight of 1,5-pentanediol, 1,4-butanediol, 1,2-cyclohexanediol and 1,4-cyclohexanediol (in each case less than 5% by weight), and also up to 5% by weight of monoalcohols, for example n-butanol, n-pentanol and n-hexanol, and less than 5% by weight of higher-boiling components than 1,6-hexanediol. For the successful purification of caprolactone in high yields, it is important that the 1,2-cyclohexanediols are removed via the top product in the distillation step.

In one embodiment of the process according to the invention, a portion of the top product from distillation step (c) is recycled into the esterification (step (a)). This variant has the advantage that the high purity of the 1,6-hexanediol stream allows the product streams in the process to be reduced in size.

In one embodiment of the process according to the invention, the distillate obtained is fed to a second column in which the fine purification of the 1,6-hexanediol is effected. This achieves 1,6-hexanediol purities of >97%.

The bottoms output of the distillation step (c) comprises the esters unconverted in the hydrogenation (step (b)), especially the esters of 6-hydroxycaproic acid and of adipic acid with the alcohol used. These esters are high boilers compared to 1,6-hexanediol.

The bottoms output of the distillation step (c) can optionally be distilled once again in order to obtain residues of hexanediol (high boiler concentration). As before, this once again affords, under analogous conditions, a 1,6-hexanediol top product (>95% 1,6-hexanediol) which may be combined with the previous top product.

The bottoms output can be analyzed more accurately by methanolysis (transesterification of all esters with methanol and analysis of the methyl esters formed) and accordingly (in the case of use of 1,6-hexanediol for DCS esterification) has the following composition:

10-40% by weight of 1,6-hexanediol (in free or esterified form), 10-40% by weight of 6-hydroxycaproic acid equivalents (6-hydroxycaproic acid is present in the bottom stream as 6-hydroxycaproic esters, for example with 1,6-hexanediol), 1-30% by weight of adipic acid equivalents (adipic acid is present in the bottom stream as adipic esters, for example with 1,6-hexanediol), 0.1-3% by weight of 1,4-cyclohexanediol, 0.1-10% by weight of bis(6-hydroxyhexyl) ether (in free or esterified form), and additionally other unidentified high boilers. 1,2-Cyclohexanediols cannot be detected in this stream.

An important factor for a high purity of ε-caprolactone is the removal of the 1,2-cyclohexanediols from the hydroxycaproic ester, since these components form azeotropes with one another. It was not foreseeable that—after the inventive combination of esterification of DCS with high-boiling alcohols (compared to ε-caprolactone) and catalytic hydrogenation of the esters formed—the 1,2-cyclohexanediols present in the DCS can be removed by distillation, together with the 1,6-hexanediol, virtually completely from the bottoms fraction comprising the 6-hydroxycaproic esters. This enables, in an economically viable manner, the preparation of ε-caprolactone with a purity of 99%, preferably of 99.9%, proceeding from DCS by cyclization of the virtually 1,2-cyclohexanediol-free (less than 0.05% by weight) 6-hydroxycaproic ester.

The bottoms fraction from step (c) is used for the cyclization (step (d)) of the 6-hydroxycaproic ester to ε-caprolactone.

The cyclization (step (d)) is effected with or without catalyst. The catalysts used may generally be acids, especially Lewis acids. Examples of such catalysts are compounds of titanium, boron, aluminum, vanadium, iron, zinc or zirconium. Preference is given to titanium tetraalkoxides Ti(OR)₄ where R represents aliphatic or aromatic radicals having 1-12 carbon atoms. The catalysts may be used in amounts between 0.01 and 1.0% by weight.

For the cyclization in the liquid phase (step (d)) of the process according to the invention, an apparatus with a column (>1 to 30 theoretical plates) is used, which can be operated continuously or batchwise. The bottom temperatures employed are 180 to 300° C., and the pressures employed between 5 and 500 mbar.

The distillate comprises generally (in the case of use of hexanediol as the esterification alcohol)40-75% hexanediol, 15-50% caprolactone, and also 1,4-butanediol, 1,5-pentanediol, 1,4-cyclohexanediol, valerolactone (in each case <5%), but no 1,2-cyclohexanediols. Remaining in the bottoms are typically especially adipic esters with hexanediol and bis(6-hydroxyhexyl)ether and other high boilers, and also the cyclization catalyst used. According to the content of utilizable adipic esters, the bottoms can be recycled or incinerated.

The bottoms output of the cyclization step (d) can be analyzed more accurately by methanolysis and accordingly (in the case of use of hexanediol as the esterification alcohol) has the following composition: 0-30% by weight of 1,6-hexanediol (in free or esterified form), 0-20% by weight of 6-hydroxycaproic acid equivalents (6-hydroxycaproic acid is present in the bottom stream as 6-hydroxycaproic esters, for example with 1,6-hexanediol), 1-60% by weight of adipic acid equivalents (adipic acid present in the bottom stream as adipic esters, for example with hexanediol), 0-1% by weight of 1,4-cyclohexanediol, 0.1-30% by weight of bis(6-hydroxyhexyl) ether (in free or esterified form), and additionally other unidentified high boilers.

According to the invention, the distillative purification of ε-caprolactone (step (e)) can be effected in one or two distillation columns. It can be effected in a batchwise process or continuously with withdrawal of the product via a side draw. In the case of use of two distillation columns, the low boilers are generally removed in the first column, and the high boilers in the second column. The distillation is effected typically in columns having 1-30 theoretical plates. The bottom temperatures employed are generally 100-250° C., and the pressures between 5 and 500 mbar. After removal of the low boilers (1,4-butanediol, 1,5-pentanediol, valerolactone), 1,2-cyclohexanediol free (less than 0.1% by weight) ε-caprolactone is obtained in good purity. The process allows the preparation of caprolactone in a purity of more than 99.9%. Remaining in the bottoms fraction are hexanediol, 1,4-cyclohexanediol and other high boilers. If required, 1,6-hexanediol can still be obtained from the bottoms.

The present invention relates to processes for preparing 1,6-hexanediol and ε-caprolactone from a dicarboxylic acid solution (DCS), comprising the steps of
a) esterification of DCS with alcohols,
b) partial catalytic hydrogenation of the ester mixture obtained from step (a),
c) distillation of the hydrogenation output obtained from step (b) to remove the 1,6-hexanediol-comprising top product, and
d) cyclization of the 6-hydroxycaproic ester from the bottoms fraction from step (c) in the presence of at least one alcohol having a boiling point greater than that of ε-caprolactone within the pressure range used, said alcohol being in free or else bound form preferably as a constituent of the esters of the bottoms fraction.

The alcohols for the esterification (step (a)) of the process according to the invention are individual alcohols having 1 to 30 carbon atoms, preference being given to those having a higher boiling point than ε-caprolactone within the pressure range from 10 to 1500 mbar (for example 96-97° C. at 20 mbar or 235° C. at 1013 mbar), and also mixtures thereof or compositions which comprise such alcohols (preferably in a proportion of at least 30% by weight, more preferably in a proportion of at least 50% by weight). Such alcohol compositions are, for example, the hydrogenation output from step (b) or the diol mixtures which can be removed as the top product in the subsequent distillation (step (c)). In this case, it is possible to use monoalcohols, and also diols and other polyhydric alcohols. Polyhydric alcohols are particularly suitable, especially diols, especially α,ω-diols having six to twelve carbon atoms. Particular preference is given to 1,6-hexanediol since this esterification alcohol corresponds to a target product of the process according to the invention. Examples of such alcohols are glycerol, trimethylolpropane, 1,6-hexanediol, 1,8-octanediol, 1,10-decanediol, 1,12-dodecanediol, 1-tridecanol, 1-pentadecanol, 1-hexadecanol, 1-octadecanol, 1-eicosanol, butylethylpropanediol, neopentyl glycol, pentaerythritol, triethylene glycol, tetraethylene glycol, bis(6-hydroxyhexyl) ether.

More particularly, the present invention relates to a process for preparing 1,6-hexanediol and very pure ε-caprolactone from a dicarboxylic acid solution (DCS), comprising the steps of
(a) esterification of DCS with alcohols,
(b) partial catalytic hydrogenation of the ester mixture obtained from step (a),
(c) distillation of the hydrogenation output obtained from step (b) to remove the 1,6-hexanediol-comprising top product, and
(d) cyclization of the 6-hydroxycaproic ester from the bottoms fraction from step (c) in the presence of at least one alcohol having a boiling point greater than that of ε-caprolactone within the pressure range used, said alcohol being in free or else bound form as a constituent of the esters of the bottoms fraction, and
   wherein alcohols having a boiling point greater than that of ε-caprolactone are introduced into the process as esterification alcohols at the early stage of the esterification step (a).

The alcohols for the esterification (step (a)) of the process according to the invention in this case are alcohols having a higher boiling point than ε-caprolactone within the pressure range from 10 to 1500 mbar (for example 96-97° C. at 20 mbar or 235° C. at 1013 mbar), and also mixtures thereof or compositions which comprise such alcohols (preferably in a proportion of at least 30% by weight, more preferably in a proportion of at least 50% by weight). Such alcohol compositions are, for example, the hydrogenation output from step (b) or the diol mixtures which can be removed as the top product in the subsequent distillation (step (c)). In this case, it is possible to use monoalcohols, and also diols and other polyhydric alcohols. Polyhydric alcohols are particularly suitable, especially diols, especially α,ω-diols having six to twelve carbon atoms. Particular preference is given to 1,6-hexanediol since this esterification alcohol corresponds to a target product of the process according to the invention. Examples of such alcohols are glycerol, trimethylolpropane, 1,6-hexanediol, 1,8-octanediol, 1,10-decanediol, 1,12-dodecanediol, 1-tridecanol, 1-pentadecanol, 1-hexadecanol, 1-octadecanol, 1-eicosanol, butylethylpropanediol, neopentyl glycol, pentaerythritol, triethylene glycol, tetraethylene glycol, bis(6-hydroxyhexyl)ether.

More particularly, the present invention relates to a process for preparing 1,6-hexanediol and very pure ε-caprolactone from a dicarboxylic acid solution (DCS), comprising the steps of
(a) esterification of DCS with low-boiling alcohols,
(b) partial catalytic hydrogenation of the ester mixture obtained from step (a),
(c) distillation of the hydrogenation output obtained from step (b) to remove the 1,6-hexanediol-comprising top product, and
(d) cyclization of the 6-hydroxycaproic ester from the bottoms fraction from step (c) in the presence of at least one alcohol having a boiling point greater than that of ε-caprolactone within the pressure range used, said alcohol being essentially 1,6-hexanediol formed in the hydrogenation in step (b) and being in free or else bound form as a constituent of the esters of the bottoms fraction, and
   wherein the hydrogenation in step (b) and/or the distillation in step (c) are performed under conditions which enable displacement of the relatively low-boiling esterification alcohols from step (a) by the 1,6-hexanediol formed in the hydrogenation in the manner of a transesterification. Such conditions are, for example, the use of hydrogenation catalyst having acidic or basic sites for the hydrogenation in step (b), the presence of small amounts of acids or bases (acid or base number in the feed to the distillation stage (c) of at least 0.01, preferably 0.01 to 5) in the distillation of step (c), and/or the presence of transesterification catalysts in the hydrogenation of step (b) and/or the distillation of step (c).

Suitable transesterification catalysts are, for example, sodium methoxide or titanates such as tetra-n-butyl titanate, in amounts based on the feed of 1 to 11300 ppm, preferably 5 to 500 ppm and more preferably 10 to 300 ppm in the hydrogenation step (b), or of 1 to 5000 ppm, preferably 5 to 3000 ppm and more preferably 10 to 12000 ppm in the distillation step (c). The alcohols for the esterification (step (a)) of the process according to the invention in this case are individual alcohols having 1 to 30 carbon atoms, preference being given to those having a lower boiling point than ε-caprolactone within the pressure range from 10 to 1500 mbar (for example 96-97° C. at 20 mbar or 235° C. at 1013 mbar), and mixtures thereof or compositions which comprise such alcohols (preferably in a proportion of at least 30% by weight, more preferably in a proportion of at least 50% by weight). Particular preference is given to methanol as the esterification alcohol.

The partial catalytic hydrogenation of the esters (step (b) of the process according to the invention) is adjusted such that a hydrogenation conversion in the range from 40 to 97% and preferably from 50 to 95% is preferably achieved.

The cyclization (step (d)) of the process according to the invention is effected in the liquid phase in an apparatus with a column having more than one theoretical plate.

The DCS of the process according to the invention is obtainable by
I) oxidation of cyclohexane with oxygen or oxygen-comprising gases to give mixtures of cyclohexanol, cyclohexanone and carboxylic acids having up to six carbon atoms, and
II) reaction of the reaction mixture obtained after step (I) with water and removal of the DCS from the liquid biphasic reaction mixture.

A preferred catalyst for the hydrogenation step (b) of the process according to the invention is obtainable by a preparation process comprising the steps of
(i) providing an oxidic material comprising copper oxide, aluminum oxide and at least one of the oxides of lanthanum, of tungsten, of molybdenum, of titanium, of zirconium or of iron,
(ii) adding pulverulent metallic copper, copper flakes, pulverulent cement, graphite or a mixture to the oxidic material from step (i) and
(iii) shaping the mixture resulting from step (ii) to a shaped body.

It was not foreseeable that such catalysts, in the case of use of oligoesters as the feed and residual acid numbers of 1 to 20 over long reaction times, would maintain a high activity coupled with high side crushing strength and hence high mechanical strength.

In one variant of the process according to the invention, the DCS used is first hydrogenated such that 5-formylvaleric acid and any 6-hydroperoxycaproic acid present therein are converted to 6-hydroxycaproic acid and 1,4-cyclohexanedione, and 4-hydroxycyclohexanone to 1,4-cyclohexanediols.

In one variant of the process'according to the invention, the 1,6-hexanediol from the top fraction of the distillation of step (c) is purified further by distillation.

In one variant of the process according to the invention, the ε-caprolactone is purified further by distillation from the distillate from step (d) (step (e)).

The processes according to the invention allow economically viable preparation of 1,6-hexanediol and very pure ε-caprolactone (purity of at least 99%, preferably of at least 99.9%) from DCS, a waste product of cyclohexane oxidation.

Since the bottom product of every process step is used as the starting material for the next step in the processes according to the invention, losses of or complex recycling of materials of value are avoided.

The process according to the invention additionally allows, to a certain degree, by alteration of the hydrogenation conversion, modulation of the product ratio between ε-caprolactone and 1,6-hexanediol.

### DESCRIPTION OF THE FIGURE

FIG. 1 illustrates the process according to the invention. The dicarboxylic acid solution (DCS) used is esterified together with preferably one high-boiling alcohol (ROH), e.g. 1,6-hexanediol (HDO), and with removal of low boilers (LB) and water (step (a)), and then catalytically partially hydrogenated with addition of hydrogen (H₂) (step (b)). Then 1,6-hexanediol (HDO), low boilers, and the 1,2-cyclohexanediols which are particularly troublesome in the later purification of ε-caprolactone, are removed from the hydrogenation output (step (c)). The 1,6-hexanediol thus removed can be purified further. The bottoms fraction of the distillation (step (c)) is processed further in the subsequent cyclization (step (d)), optionally after addition of a cyclization catalyst. ε-Caprolactone (CLO) is obtained from the top fraction, which can be purified further by distillation (step (e)) with removal of the low boilers (LB) and high boilers (HB) to give very pure c-caprolactone (CLO). The high boiler fraction (HB) from step (d) can optionally be recycled into the hydrogenation step (b). Parts of the alcohol-containing fraction from step (b) and/or (c) can, optionally after further purification, be used for the esterification of the DCS in step (a).

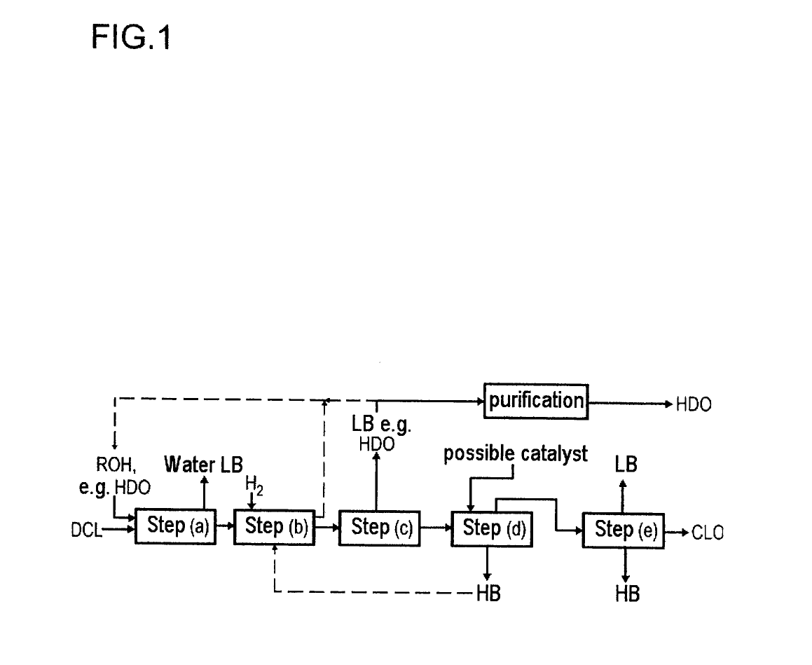

### WORKING EXAMPLES

### Example 1

### 1. Blending of a Dicarboxylic Acid-Hexanediol Mixture

The dicarboxylic acid solution (DCS) used was obtained by aqueous extraction of a reaction discharge which originated from the oxidation of cyclohexane with air. The 1,6-hexanediol-containing diol mixture used for the esterification was prepared by distillative removal of high and low boilers (compared to the diols) from the hydrogenation output of the oligo- and polyester hydrogenation (see stages 4a and 4b).

To 209 kg of a DCS (acid number: 268 mg KOH/g) comprising, inter alia, adipic acid (ADA, 21.6% by weight), 6-hydroxycaproic acid (HCS, 14.5% by weight) and water (48% by weight), partly in the form of oligomers (oligohydroxycaproic acid, oligomers consisting of adipic acid and hydroxycaproic acid units), were added 94 kg of a 1,6-hexanediol-containing diol mixture. The hexanediol-containing diol mixture comprised, inter alia, 1,6-hexanediol (approx. 83% by weight), 1,5-pentanediol (approx. 8% by weight), and also 1,4-butanediol, 1,4-cyclohexanediol and 1,2-cyclohexanediol (in each case <2% by weight).

### 2. Preparation of an Oligomeric Ester Mixture

### Process Step (a)

The DCS-hexanediol mixture of stage 1 was metered continuously into an evaporator (dewatering stage, 150° C., ambient pressure) at a throughput of 275 g/h. This distilled off water and low-boiling components (127 g/h). The bottoms output was subsequently transferred to a 5-stage stirred tank cascade (180 g/h, 220° C., 1-1.4 bar abs.), in which esterification was brought to virtual completion (acid number: 6 mg KOH/g, corresponding to 98% conversion). In the ester cascade, low-boiling components were likewise distilled off (14 g/h), and were recycled into the dewatering stage. The bottoms output obtained was an oligomeric mixture comprising principally esters of the originally supplied carboxylic acid derivatives and diols (156 g/h, 57% yield by weight, based on the overall feed, ester number: 348 mg KOH/g).

### 3. Hydrogenation of the Oligomeric Ester Mixture

### Process Step (b)

The oligomeric esters of stage 2 were admixed with 60 ppm of sodium methoxide and then hydrogenated continuously over a copper catalyst. The catalyst was prepared and activated according to WO 2007/6719 example 3.

The reactor system consisted of a main reactor (tubular reactor, 400 mL, 600 g of catalyst) and a postreactor (tubular reactor, 100 mL, 150 g of catalyst). The hydrogenation feed was passed over the fixed bed catalyst in trickle mode. In order to remove the heat which evolves in the hydrogenation, the main reactor was operated with liquid circulation, and the postreactor in straight pass.

The hydrogenation reactor was operated at 240° C. and 255 bar of H₂ for 600 h. At a feed rate of 250 g/h (catalyst hourly space velocity: 0.63 kgL⁻¹ h⁻¹, main reactor), a conversion of 93% was achieved (ester number: 24 mg KOH/g). The hydrogenation output was subsequently decompressed to ambient pressure in a vessel and cooled to ambient temperature. This afforded outputs whose 1,6-hexanediol contents were 71% by weight. The hydrogenation proceeded with 92% yield to 1,6-hexanediol (the yield relates to the O₆ components which are present in the DCS and can lead to 1,6-HDO by hydrogenation: 6-hydroxycaproic acid, 6-oxocaproic acid (5-formylvaleric acid), adipic acid and dihydromuconic acid).

The 1,6-hexanediol content was determined by gas chromatography: DB-5 (Agilent J&W), 30 m×0.2 mm×1 μm; temperature profile: 60° C. (5 min) to 220° C. (16° C./min, 10 min) to 260° C. (20° C./min, 21 min) to 290° C. (20° C./min, 10 min). Diethylene glycol dimethyl ether (DEGDME) was used as an internal standard, tR (DEGDME)=8.8 min, 40 tR (1,6-hexanediol)=11.8 min.

4a. Removal of High Boilers from the Hydrogenation Output (Process Step (c))

In a distillation still with an attached column (DN50, internals 1 m, fabric packing 750 m²/m³), hydrogenation outputs (38 kg) from stage 3 were separated by distillation. At 50 mbar and bottom temperature 178° C., 32 kg of top product were obtained (reflux ratio 1:1), which comprised 81% by weight of 1,6-hexanediol (additionally 8% by weight of pentanediol, 2% by weight of 1,2-cyclohexanediol, 1.4% by weight of 1,4-cyclohexanediol, others 7% by weight). Additionally obtained were 6 kg of bottom product which, according to gas chromatography, comprises 33% by weight of 1,6-hexanediol, and also adipic and hydroxycaproic esters (not quantified).

4b. High Boiler Concentration

In a distillation still with attached column (DN50, internals 1 m, fabric packing 750 m²/m³), the bottoms outputs (1.7 kg) from stage 4a were concentrated by distillation. This afforded, at 20 mbar and a bottom temperature of 220° C. (reflux ratio 17:1), 0.54 kg of top product which had a hexanediol content of >95%. The bottom product obtained was 1.16 kg of concentrated high boilers. According to gas chromatography, this stream also comprised 9% hexanediol. The composition was determined more accurately by methanolysis of this stream: for this purpose, 15 g of the bottoms output were admixed with 150 ml of methanol and 0.05 g of tetra-n-butyl titanate, and heated to 170° C. in a 300 ml autoclave for 6 hours. The 1,6-hexanediol (HDO), methyl 6-hydroxycaproate (MeHC), dimethyl adipate (DMeAD), 1,2-cyclohexanediol (1,2-CHDO) and 1,4-cyclohexanediol (1,4-CHDO) contents were determined by gas chromatography (DB-5, 30 m×0.23 mm×1 μm, 60° C. (5 min) to 220° C. (16° C./min, 10 min) to 260° C. (20° C./min, 10 min); diethylene glycol dimethyl ether as an internal standard, retention time 1,2-CHDO: 10.8 min; 1,4-CHDO: 11.1 and 11.2 min, HDO: 11.5 min; MeHC: 12.1 min; DMeAD: 12.6 min; DiHDO: 19.2 min). Accordingly, the sample used comprised 22.7% by weight of HDO, 14.2% by weight of 6-hydroxycaproic acid (HCA), 8.9% by weight of adipic acid (ADA), 0.6% by weight of 1,4-CHDO and 7.2% by weight of DiHDO. 1,2-CHDO was undetectable in this fraction.

### 5. Cyclization

### Process Step (d)

The bottom stream obtained in stage 4b (501 g) was introduced into the bottoms vessel of a distillation column (30 cm, filled with 5×5 mm glass Raschig rings) and admixed with 1 g of tetra-n-butyl titanate. At 30 mbar, a bottom temperature of initially 180° C. was established, which was increased continuously up to 270° C. with increasing distillation time (return ratio 50:10). A total of 198 g of top product were obtained, which comprised the following components: 4% by weight of pentanediol, 1% by weight of 8-valerolactone, 1% by weight of 1,4-cyclohexanediol, 55% by weight of 1,6-hexanediol, 30% by weight of ε-caprolactone). This corresponds to an caprolactone yield of 97% based on the hydroxycaproic acid units present in the stream used, and a 1,6-hexanediol yield of 96% based on the hexanediol units used. No 1,2-cyclohexanediols were detectable in the distillate.

271 g of bottoms output were obtained. According to methanolysis (analogously to stage 4b), this output also comprised 0.5% by weight of 1,6-hexanediol, 0.4% by weight of hydroxycaproic acid, 13.9% by weight of adipic acid and 12.5% by weight of bis(6-hydroxyhexyl)ether. 1,2- and 1,4-Cyclohexanediols were undetectable in this fraction.

### 6. ε-Caprolactone Purifying Distillation

The ε-caprolactone-comprising distillate obtained in stage 5 was introduced into the bottoms vessel of a distillation column (1 m, filled with 5×5 metal Raschig rings). The mixture was performed at 10 mbar and a bottom temperature of 145° C. (return ratio 20:10). After removal of 30 g of first runnings (ε-caprolactone content of 20% by weight), the main fraction was taken at a bottom temperature of 150-155° C. (41 g, ε-caprolactone content 99.9%, remainder valerolactone). Subsequently, the distillation was ended. If required, it would also be possible to distil off the hexanediol remaining in the bottoms.

### Example 2

Comparative example analogous to the process described in EP 883591 A (“variant E”): cyclization from a DCS stream esterified with hexanediol.

707 g of an ester mixture obtained analogously to example 1 stage 2 (according to methanolysis 0.2% by weight of 1,2-cyclohexanediol, 1.6% by weight of 1,4-cyclohexanediol, 45.4% by weight of hexanediol, 17.7% by weight of 6-hydroxycaproic acid equivalents, 20.0% by weight of adipic acid equivalents) were introduced into the bottoms vessel of a 1 m distillation column (filled with 5×5 mm wire mesh rings) and distilled without addition of titanate at 20 mbar and a bottom temperature between 200 and 246° C. (return ratio 10:1). 65 g of a distillate were obtained, which had the following composition: 1.0% by weight of 1,2-cyclohexanediols, 5.4% by weight of 1,4-cyclohexanediols, 22% by weight of hexanediol, 44% by weight of caprolactone. In addition, a series of unidentified by-products were also detected.

In the first distillation stage, 46% of the 1,2-cyclohexanediols used, 31% of the 1,4-cyclohexanediols used, 4% of the hexanediol and 26% of the caprolactone were thus distilled off.

0.5 g of tetra-n-butyl titanate was added to the remaining bottoms, and distillation was effected again at 20 mbar and a bottom temperature between 210 and 255° C. (return ratio 10:1). This afforded a total of 154 g of a distillate which had the following composition: 0.4% by weight of 1,2-cyclohexanediols, 2.1% by weight of 1,4-cyclohexanediols, 29% by weight of hexanediol, 44% by weight of caprolactone. In addition, a series of unidentified by-products were also detected.

In the second distillation stage, 44% of the amount of 1,2-cyclohexanediol, 29% of the amount of 1,4-cyclohexanediol, 14% of the amount of hexanediol and 63% of the amount of caprolactone were thus distilled off.

It is thus impossible here to distillatively remove the 1,2-cyclohexanediols before the actual caprolactone production so as to achieve a high purity of caprolactone.

1. A process for preparing 1,6-hexanediol and ε-caprolactone, comprising the following steps:
a) esterification of DCS with an alcohol,
b) partial catalytic hydrogenation of the ester mixture obtained from step (a),
c) distillation of the hydrogenation output obtained from step (b) to remove the 1,6-hexanediol-comprising top product, and
d) cyclization of the 6-hydroxycaproic ester from the bottoms fraction from step (c) in the presence of at least one alcohol having a boiling point greater than that of ε-caprolactone within the pressure range used, a mixture of such alcohols or a composition which comprises such alcohols, said alcohol being in free or else bound form as a constituent of the esters of the bottoms fraction.

2. The process according to claim 1, wherein the esterification in step (a) is performed with an alcohol having a higher boiling point than ε-caprolactone within the pressure range from 10 to 1500 mbar, a mixture thereof or a composition which comprises such alcohols.

3. The process according to claim 1 or 2, where the alcohols used for the esterification in step (a) are polyhydric alcohols, especially diols.

4. The process according to claim 2 or 3, wherein a portion of the hydrogenation output from step (b) is used as the alcohol-comprising composition for the esterification of the DCS in step (a).

5. The process according to any of claims 2 to 4, wherein a portion of the alcohol-comprising composition which is obtained as the top product from the distillation in step (c) is used for the esterification of the DCS in step (a).

6. The process according to any of claims 2 to 5, wherein 1,6-hexanediol is used for the esterification of the DCS in step (a).

7. The process according to claim 1, wherein the esterification in step (a) is performed with an alcohol having a lower boiling point than ε-caprolactone within the pressure range from 10 to 1500 mbar, a mixture of such alcohols or a composition which comprises such alcohols, and
wherein the hydrogenation in step (b) and/or the distillation in step (c) are performed under conditions which enable displacement of the relatively low-boiling esterification alcohol from step (a) by the 1,6-hexanediol formed in the hydrogenation in the manner of a transesterification.

8. The process according to claim 7, wherein the transesterification-enabling conditions are the use of a hydrogenation catalyst having acidic or basic sites in step (b), the presence of acids or bases in step (c) in an amount which causes an acid or base number of at least 0.01 in the feed to the distillation stage, and/or the presence of trans-esterification catalysts in step (b) and/or (c) in amounts of at least 1 ppm based on the feed.

9. The process according to any of claims 1 to 8, wherein the partial hydrogenation in step (b) is adjusted such that a hydrogenation conversion in the range from 40 to 97% is achieved.

10. The process according to any of claims 1 to 9, wherein the DCS is obtained by
I) oxidation of cyclohexane with oxygen or oxygen-comprising gases to give mixtures of cyclohexanol, cyclohexanone and carboxylic acids having up to six carbon atoms, and
II) reaction of the reaction mixture obtained after step (I) with water and removal of the DCS from the liquid biphasic reaction mixture.

11. The process according to any of claims 1 to 10, wherein the cyclization in step (d) is performed in the liquid phase in an apparatus with a column having more than one theoretical plate.

12. The process according to any of claims 1 to 11, wherein the 1,6-hexanediol from the top fraction of the distillation of step (c) is purified further by distillation.

13. The process according to any of claims 1 to 12, wherein the ε-caprolactone is purified further from the distillate from step (d) in a subsequent distillation step (e).

14. The process according to any of claims 1 to 13, wherein the alcohol-comprising composition consists of the corresponding alcohols in a proportion of at least 30% by weight.

15. The process according to any of claims 1 to 14, wherein the hydrogenation in step (b) of the esterification mixture from step (a) is hydrogenated in the liquid phase in the presence of a shaped catalyst body, the precursor of which is obtainable by
(i) providing an oxidic material comprising copper oxide, aluminum oxide and at least one of the oxides of lanthanum, of tungsten, of molybdenum, of titanium, of zirconium or of iron,
(ii) adding pulverulent metallic copper, copper flakes, pulverulent cement, graphite or a mixture to the oxidic material from step (i) and
(iii) shaping the mixture resulting from step (ii) to a shaped body.

16. The process according to any of claims 1 to 15, wherein the esterification in step (a) is performed without addition of catalyst.

17. The process according to any of claims 1 to 16, wherein 1,2-cyclohexanediols are removed via the top in step (c).
